(19) United States
(12) Patent Application Publication
(10) Pub. No.: US 2013/0237789 A1
(43) Pub. Date: Sep. 12, 2013

(54) ELECTRODE AND METHOD FOR PRODUCING SUCH AN ELECTRODE

(71) Applicant: Giorgio Cattaneo, Karlsruhe (DE)

(72) Inventor: Giorgio Cattaneo, Karlsruhe (DE)

(73) Assignee: ACANDIS GMBH & CO. KG, Pfinztal (DE)

(21) Appl. No.: 13/696,491

(22) PCT Filed: May 6, 2011
(86) PCT No.: PCT/EP2011/002263

§ 371 Date: Jan. 17, 2013

(30) Foreign Application Priority Data
May 6, 2010 (DE) 10 2010 019 648.7

(51) Int. Cl. A61N 1/04; A61B 18/14; A61B 5/04

(52) U.S. Cl. CPC A61N 1/04; A61B 5/04; A61B 18/14
USPC 600/372; 607/115; 606/41; 204/192.1; 427/2.11

(57) ABSTRACT

An electrode for measuring electrical activities of a living body and/or for outputting electrical signals into the living body, with an electrically insulated and flexible line, which is connected fixedly or releasably to a distally arranged and electrically conductive electrode tip. The electrode tip is designed for insertion into the body. The electrode tip includes at least one crystalline shape-memory material, and the electrode tip is flexible on account of the shape-memory material. The shape-memory material of the support structure is formed at least partially by physical vapour deposition, in such a way that precipitations in the shape-memory material have a maximum size of 500 nm.

The invention relates to an electrode, in particular for measuring electrical activities of a living body and/or for outputting electrical signals into the living body, with the features of the preamble of claim 1. The invention further relates to a method for producing such an electrode.

An electrode of the aforementioned type is known, for example, from DE 199 30 271 A1.

Electrodes are used in the medical sector both for outputting electrical signals into the living body and also for measuring electrical activities of body cells. The measurement or the stimulation of body cells can take place inside the body or outside the body, for example on the skin surface. Electrodes for medical purposes can be provided for permanent implantation, for temporary implantation or for temporary contact with the tissue. For example, it is known to guide electrodes to a measurement site via a catheter and, after the measurement or stimulation has taken place, to remove the electrodes. It is necessary in all cases to ensure the greatest possible flexibility of the electrode, so that the treatment sites or measurement sites inside or outside the body can be easily reached. In particular, it is desirable to establish secure contact between the electrode and the body tissue.

The known electrode mentioned above has an electrode tip, which comprises an electrically conductive surface. The electrode tip is connected to an electrically insulated line. During use, the electrode tip is in contact with body tissue, such that electrical signals or electrical impulses can be transferred to the body tissue. The known electrode is provided in particular for implantation in the heart, in order to transfer electrical signals of an implanted pacemaker or defibrillator to the cells of the heart muscle. The connection between the device, which generates the electrical signals, and the electrode tip is established via the electrically insulated line, which is guided inside blood vessels to the treatment site. The electrically insulated line is flexible, so as to be able to follow the curves and branches of blood vessels. As regards the material of the electrode tip, it is merely disclosed that the latter is made of metal.

The known electrode has the disadvantage that the electrode tip is rigid or at least only partially flexible. Therefore, when the electrode tip anchors in the heart muscle, relatively large tissue regions are inhibited in terms of their muscle movement.

The object of the invention is to make available an electrode for measuring electrical activities of a living body and/or for outputting electrical signals into the living body, which electrode has a high degree of flexibility and allows electrical signals to be transferred, in particular transmitted and received, in a reliable manner. It is also the object of the invention to make available a method for producing such an electrode.

According to the invention, this object is achieved, in terms of the flexible electrode, by the subject matter of claim 1, and, in terms of the method, by the subject matter of claim 11.

The invention is based on the concept of making available an electrode for measuring electrical activities of a living body and/or for outputting electrical signals or impulses into the living body and/or for ablating tissue or nerve paths or the system of stimulus conduction in the heart, with an electrically insulated and flexible line which is connected fixedly or releasably to a distally arranged and electrically conductive electrode tip, the electrode tip being designed for insertion into the body. At least one support structure of the electrode tip comprises at least one crystalline shape-memory material. The electrode tip is flexible on account of the shape-memory material. The shape-memory material of the support structure is formed at least partially by physical vapor deposition, in such a way that precipitations in the shape-memory material have a maximum size of 500 nm.

A shape-memory material is understood as a material that has the property whereby after a change of shape in the martensitic state by heating into the starting state it is able to partially or almost completely recover its original shape. The martensite formation can also take place through mechanical stressing at a temperature above the austenite finish temperature (stress-induced martensite).

The advantage of the crystalline shape-memory material is that a pseudo-elastic change of shape of the support structure of the electrode tip can be achieved through the shape-memory material, which change of shape takes place in a manner known per se by formation of stress-induced martensite. The flexibility of the support structure is to this extent effected by the shape-memory material. The measures needed to adjust the pseudo-elastic properties of the shape-memory material are known to a person skilled in the art. By virtue of the pseudo-elasticity of the support structure of the electrode tip, and by virtue of the associated flexibility, it is possible for the entire electrode, including the electrically insulated and flexible line connected fixedly or releasably to the flexible electrode tip, to be easily adapted to different shapes of vessels or organs.

The support structure of the electrode tip forms the area of the electrode tip that performs a supporting function and that is designed to take up and convey mechanical forces, for example on account of muscle movements. In addition, the support structure or the shape-memory material, in particular the metallic shape-memory material, is electrically conductive. The coating of a conventionally produced support structure with another material, for example with gold, is not regarded as part of the support structure itself if the coating does not contribute to the mechanical properties of the support structure, i.e. does not perform a supporting function.

According to the invention, provision is also made that the shape-memory material of the support structure is formed at least partially by physical vapor deposition, in such a way that precipitations in the shape-memory material have a maximum size of 500 nm. In this way, the material of the support structure differs structurally from electrode tips that are produced conventionally from solid material, for example usually by reshaping. The upper limit of the size of the precipitations can be 450 nm, in particular 400 nm, in particular 350 nm, in particular 300 nm, in particular 250 nm, in particular 200 nm, in particular 150 nm, in particular 100 nm, in particular 75 nm, in particular 50 nm, in particular 25 nm, in particular 20 nm. The lower limit can be 10 nm, in particular 15 nm, in particular 20 nm, in particular 25 nm, in particular 30 nm, in particular 35 nm, in particular 40 nm, in particular 45 nm, in particular 50 nm. The aforementioned lower limits can be combined with the aforementioned respectively higher upper limits.

The conventional production of electrode tips comprises vacuum fusion of the starting material, hot-forming of the material, for example by swaging and/or hot rolling, and cold-forming, e.g. for producing a pipe or wire in many individual steps. This is often followed by further processing steps, e.g. by means of a laser, and formative heat treatments, and finally by finishing, e.g. by electropolishing. By contrast, in vapor deposition, in particular in sputtering technology, target material is melted and poured off in the vacuum. The target material thus produced is atomized and deposited on a substrate (sputtering). By subsequent heat treatment, the amorphous material is converted into crystalline, superelastic material. Alternatively, the deposition of the material can take place at high temperatures in such a way that, during deposition, the material crystallizes out, for example on a heated substrate.

In the conventional production of the shape-memory material by melting, inclusions are inevitably formed, for example TiC and Ti₂NiOₓ. These inclusions occur in the melt, grow during the cooling of the melt and are then present in the hardened material. The inclusions caused by the melting generally contain titanium, nickel and also carbon and/or oxygen. The size of the inclusions ranges from a few μm to more than 20 μm. On account of the melt phase, such inclusions of this size occur only in conventionally produced material.

In the context of the application, inclusions are therefore understood as phases which are formed by melting and in which foreign atoms, for example carbon or oxygen, may be present which, during the production and processing of the material, are introduced from the outside and are therefore not precipitated from the alloy structure.

By contrast, material produced by thin-film technology or vapor deposition, in particular sputtering, has no inclusions, i.e. is free of inclusions, because the material is not produced directly from a melt but instead by deposition of the atomized target material on a substrate. This may also possibly be due to the fact that the oxygen and carbon contents achievable by vapor deposition, in particular by sputtering, are lower than in materials that have been produced conventionally by melting, since the vapor deposition and in particular the sputtering takes place in vacuum. However, in the event of inclusions being caused by contaminants, these inclusions are much smaller in support structures produced by deposition than in support structures produced by melting. Any inclusions have a size of less than 5 μm, in particular less than 3 μm, in particular less than 1 μm, in particular less than 500 nm, in particular less than 200 nm, in particular less than 100 nm, in particular less than 50 nm, in particular less than 20 nm, in particular less than 10 nm. In conventionally produced material, in addition to the inclusions, the heat treatment can cause precipitations (or precipitates) of phases from the solid solution, which grow as a result of the multiple annealing treatments and/or as a result of the reshaping of the material.

On account of the support structure being produced by vapor deposition, in particular by sputtering, the precipitations (or precipitates) that are present according to the invention in the shape-memory material of the support structure differ in size from the inclusions caused by melting, being smaller by several orders of magnitude than the inclusions caused by melting (maximum size of the precipitations: 500 nm).

In terms of their origin, precipitations differ from inclusions in that precipitations form as phase from the solid solution, i.e. from the crystalline material, i.e. are precipitated. Inclusions form in the melt as a result of nucleation growth and remain, or are enclosed, in the hardened material. From the different size of the precipitations/inclusions in the structure, it is possible to see by which method the shape-memory material of the support structure was produced. The same applies to possible precipitations that may occur in support structures produced by melting and that are larger than precipitations in support structures that are produced by deposition.

Precipitations consist of the alloy components and generally contain no foreign atoms introduced from outside, since the production process in vapor deposition is cleaner compared to the conventional process. For example, precipitations in shape-memory materials made of NiTi alloys generally consist of titanium and of nickel, with different types of precipitation resulting from different compositions. Typical precipitations that are obtainable by deposition comprise Ni₄Ti₃.

A further difference in the material revealing the different production methods is that the inclusions and precipitations are arranged in rows in the direction of pulling (pipe or wire) or the direction of rolling (sheet metal), whereas the precipitations of the shape-memory material of the support structure according to the invention have an isotropic orientation. The isotropic, that is to say random, orientation of the precipitations has a positive effect on conductivity.

According to the invention, the conductivity is substantially homogeneous in the electrode tip, particularly in the support structure, and more homogeneous than in conventional electrode tips. This is because conductivity, in addition to being dependent on temperature inter alia, is also influenced by inclusions, where the charge carriers are scattered. On account of the small size of the precipitations of the shape-memory material of the support structure according to the invention, the scattering is reduced and conductivity improved. In this way, the electrode according to the invention is suitable particularly, but not exclusively, for use in sensor systems.

Furthermore, the support structure of the electrode tip performs a dual function. On account of the pseudo-elastic effect of the shape-memory material, the electrode tip has, on the one hand, good mechanical properties, in particular good flexibility. On account of the small size of the precipitations, the support structure can be expected to have improved fatigue behavior. On the other hand, the electrical properties of the shape-memory material, in particular the conductivity, are improved as a result of the maximum size of the precipitations from physical vapor deposition.

Overall, in conventionally produced electrodes, the many successive processes (melting and hardening, repeated hot-forming and cold-forming with intermediate heat treatments, in particular intermediate annealing) result in an undefined number of lattice defects, which are not controllable. These lattice defects interact with one another. For example, the density of the voids influences the dislocation formation and the dislocation movement. Cold-forming clearly causes the dislocation formation and, together with the annealing treatments, also greatly influences the grain size. Thus, in a conventionally produced component, it is not possible to exactly predict or adjust the electrical resistance, nor is it possible to avoid solder-to-solder differences. Moreover, in a conventional electrode consisting of many individual contacts, the individual contacts can have different electrical resistances. In addition, the final components, in particular NiTi components, such as the electrode or electrode tip, which are produced from conventional pipe or wire, have different electrical properties over the cross section of the component, and often along the length thereof, because the lattice defects are differently distributed over the cross section and/or the length of the component.

By contrast, an electrode tip made from a shape-memory material, or the support structure thereof, which is produced by a PVD method, in particular by sputtering, is purer, i.e. contains less carbon and less oxygen, than conventional material. The number of the lattice defects (voids, dislocations) is smaller than in conventional material and, including the grain boundaries, can be better controlled on account of the lesser external damage (introduction of contaminants). The reason for this is that, in sputtering, only the starting material is melted. Thereafter, there is only the sputtering process and a single annealing treatment. The range of fluctuation of the density of voids, dislocations or grain boundaries over the cross section of the electrode is small. Inclusions are avoided.

Preferably, the shape-memory material of the support structure has a maximum density, specifically adjusted by physical vapor deposition (PVD), of material inhomogeneities such as lattice defects and/or boundary defects and/or volume defects. In this way, the material of the support structure further differs from electrode tips which are produced conventionally from solid material, for example by reshaping, and which have a higher density of material inhomogeneities than do electrode tips that are produced by physical vapor deposition (PVD). Defects in the structure of the material, i.e. material inhomogeneities such as lattice defects (for example voids, interstitial atoms, edge dislocation, screw dislocation, stacking faults) and/or boundary defects and/or volume defects, likewise impede the charge transport and reduce conductivity. Because the density of material inhomogeneities is limited by the physical vapor deposition to a maximum value, the reduction in conductivity is further limited, and an electrode tip is obtained with a uniform conductivity that is advantageous for sensor systems.

The material inhomogeneities concern, for example, dislocations, grain boundaries and inclusions in the metal lattice. These zero-dimensional and/or unidimensional and/or two-dimensional and/or three-dimensional lattice defects are controlled in the production of the flexible electrode, in particular of the electrode tip.

Preferably, the mean grain size of the shape-memory material of the support structure is less than 4 μm, in particular less than 3 μm, in particular less than 2 μm, in particular less than 1.5 μm, in particular less than 1 μm, in particular less than 0.5 μm, in particular less than 250 nm. In contrast to this, average grain sizes in conventionally produced electrodes are from 5 μm to 20 μm. Moreover, support structures that are produced by deposition techniques have less variation in grain size than does conventional material, both within a component and also from solder to solder. The grain size is homogeneous, in particular more homogeneous than in material that is produced by melting techniques. The conductivity is further improved by the homogeneous average grain size.

In order to influence or adjust the size of the precipitations and the density of inhomogeneities such as linear lattice defects and/or boundary defects and/or volume defects, provision is advantageously made that the electrode tip is produced at least partially by sputtering or by laser-assisted deposition. By varying individual process parameters of the vapor deposition method, it is possible to specifically adjust the inhomogeneities in the material of the electrode tip. Suitable process variables are, for example, the chamber pressure and/or the deposition pressure and/or the partial pressure of the process gas and/or the target composition.

By variation of parameters of this kind, it is possible in a manner known per se to specifically adjust the size of the precipitations or of the lattice defects and the density thereof. Moreover, the grain size can also be specifically adjusted. By test methods using an electron microscope, in particular using a scanning electron microscope or a transmitting electron microscope, it is possible to monitor the specifically adjusted inhomogeneities or lattice defects. The quality and quantity of the measured or detected lattice defects can be used to adjust the process parameters of the vapor deposition method.

The use of shape-memory materials is known per se in medicine. As has been explained above, the production of components from shape-memory materials usually involves vacuum fusion, hot-forming, cold-forming and subsequent shaping. In this production method, structural transitions bring about volume defects, for example lattice voids and large inclusions, which are not controllable.

Lattice defects of this kind are reduced or avoided by using the physical vapor deposition method. This increases the homogeneity of the electrical conductivity of the electrode tip and, therefore, the precision of the sensor system. Moreover, the specifically adjusted density of lattice defects in the material of the electrode tip ensures a uniform quality of the electrodes produced, not just within one production batch, but also between different batches. The rejection rate is thereby reduced.

The shape-memory material preferably comprises a nickel-titanium alloy, in particular with a composition of 50.8 atomic percent nickel and 49.2 atomic percent titanium. The nickel-titanium alloy is particularly suitable for achieving the increased flexibility of the electrode tip, in particular the pseudo-elastic properties. In the specific composition indicated, lattice defects are further reduced, such that the conductivity of the electrode tip can be positively influenced.

The shape-memory material can comprise a ternary or quaternary alloy. Such alloys are easy and inexpensive to produce.

In a preferred embodiment of the flexible electrode according to the invention, the electrode tip has a multi-layer configuration. By means of the multi-layer configuration of the electrode tip, several different electrical signals can be output into the living body or received from the living body. The electrode tip composed of several layers can therefore form a combined stimulation electrode tip and detection electrode tip. The stimulation electrode tip is designed to output energy into the body tissue, and the detection electrode tip is designed to pick up signals from the body tissue. For example, the multi-layer electrode tip can be used to detect electrical activities of body cells, for example of heart muscle cells, and at the same time to stimulate the same body cells. Other applications are possible, for example on nerve paths.

Preferably, a radially outer layer or several radially outer layers are composed of the shape-memory material, in particular of a nickel-titanium alloy, and a radially inner layer or several radially inner layers are composed of a material with higher electrical conductivity than the material of the radially outer layer or radially outer layers. In particular, the material of the radially inner layer or of the several radially inner layers is preferably composed of copper, silver, gold, platinum, tantalum, niobium, palladium or carbon. In the case of a cylindrical electrode tip, the radially outer layer or the radially outer layers are stressed further by torsion or bending of the electrode tip. The pseudo-elastic properties provided by the shape-memory material are particularly advantageous in these outer layers, since in this way the flexibility of the electrode tip is increased. Using a material with higher electrical conductivity in one or more radially inner layers ensures that electrical signals can be efficiently output into the body or received from the body through the flexible electrode tip.

A radially continuous transition can be present between the layers. The electrode tip can thus substantially correspond to the configuration of a graded-index fiber, with a material gradient being provided between the radial outer circumference of the electrode tip and the longitudinal axis of the electrode tip. The material of the outer layers and the material of the inner layers can run into each other or merge smoothly into each other.

The radially inner layer or the radially inner layers can be composed of a nickel-titanium alloy and the radially outer layer or the radially outer layers can be composed of a material with higher electrical conductivity than the material of the radially inner layer or the radially inner layers. Such a layered construction is advantageous if electrical signals are to be transferred along the length of the electrode tip, in particular over the outer circumference of the cylindrical electrode tip.

According to a further aspect, the invention is based on the concept of making available a method for producing an electrode for measuring electrical activities of a living body and/or for outputting electrical signals into the living body. The electrode has an electrically insulated and flexible line which is connected fixedly or releasably to a distally arranged and electrically conductive electrode tip, the electrode tip being designed for insertion into the body. In the method, at least one support structure of the electrode tip is formed at least partially from at least one crystalline shape-memory material by physical vapor deposition, in such a way that the electrode tip is flexible on account of the shape-memory material.

The physical vapor deposition can take place, for example, in the form of sputtering or as laser-assisted deposition. Sputtering, or cathodic sputtering, in particular magnetron sputtering is highlighted as being a particularly suitable method. PVD methods of this kind are known per se to a person skilled in the art.

The method according to the invention has the advantage that the maximum density of material inhomogeneities, such as lattice defects, in the electrode tip can be specifically adjusted, for example by variation of suitable process parameters, such as chamber pressure and/or deposition pressure and/or partial pressure of the process gas and/or target composition. It is thus possible, in a particularly simple way, to make available a flexible electrode tip that has improved properties in respect of conductivity. The production method according to the invention also ensures a uniform quality of the electrodes produced, not only within one batch but also between several batches. In particular, the rejection rate can be reduced using the production method according to the invention.

In a particular embodiment of the method according to the invention, provision is made that several layers of different materials are applied for forming the electrode tip, wherein the radially inner layers and the radially outer layers are produced by a combination of a lithography method and a sputtering method. With the aid of the lithography method, the inner and/or outer layers of the electrode tip can be structured, such that particular surface structures or geometric arrangements of the different materials can be achieved. By combining the lithography method with the sputtering method, different geometries or arrangements of the layered structure of the electrode tip can be easily produced. On account of the short change-over times when using the lithography method and the sputtering method, it is possible to produce different electrodes within a short time, each with a different geometric arrangement of the various materials.

The method also allows printed conductors and circuits to be integrated in the electrode tip by sputtering. A corresponding production method and a corresponding electrode tip, or an electrode with a corresponding electrode tip, are to this extent disclosed.

The advantages and effects mentioned in connection with the above-described electrode also apply in respect of the production method and are likewise disclosed and claimed in this connection. The same applies to the process steps described in connection with the electrode.

The invention is explained in more detail below on the basis of illustrative embodiments and with reference to the attached schematic drawings, in which:

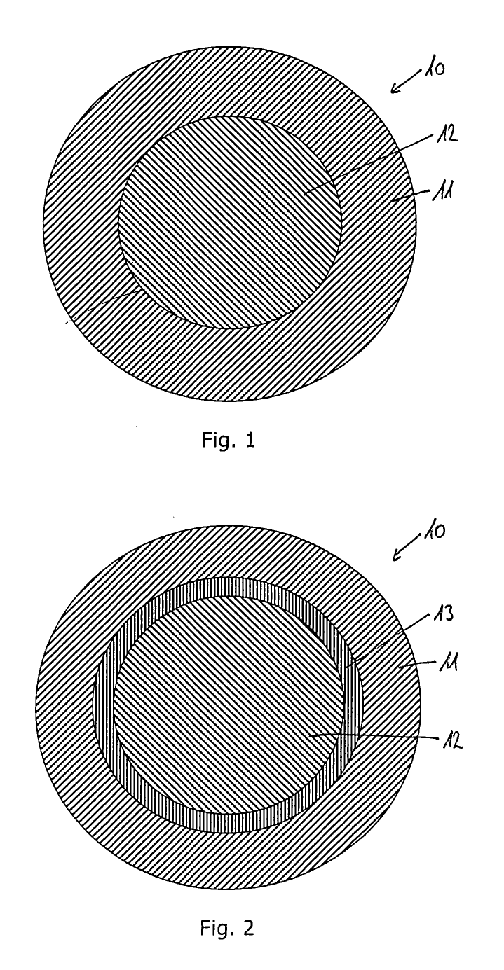

FIG. 1 shows a cross section through an electrode tip of an electrode according to the invention, in a preferred illustrative embodiment;

FIG. 2 shows a cross section through an electrode tip of an electrode according to the invention, in another preferred illustrative embodiment;

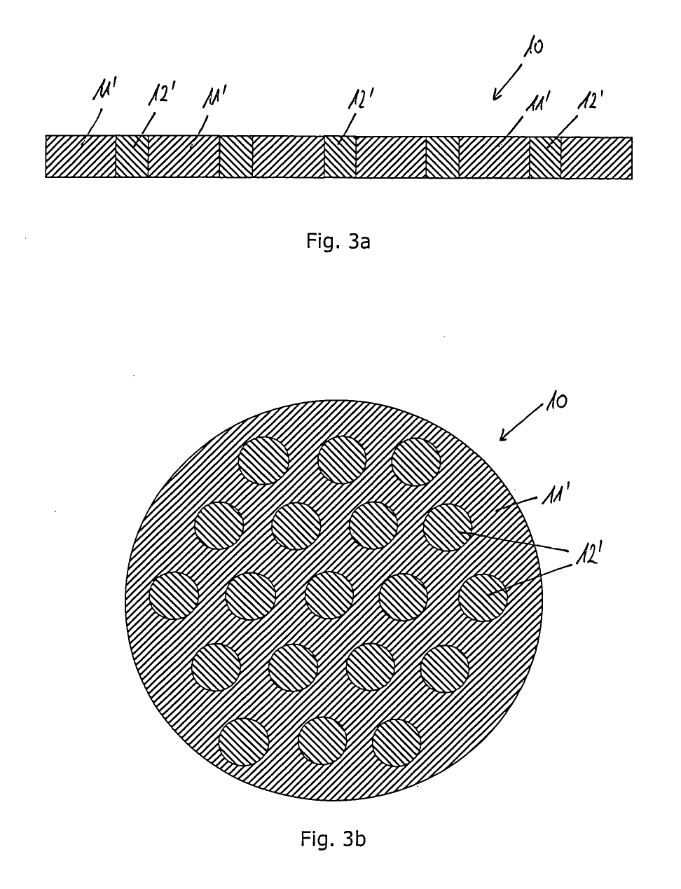

FIG. 3a shows a cross section through an electrode tip of an electrode according to the invention, in another preferred illustrative embodiment;

FIG. 3b shows a plan view of the electrode tip according to FIG. 3a;

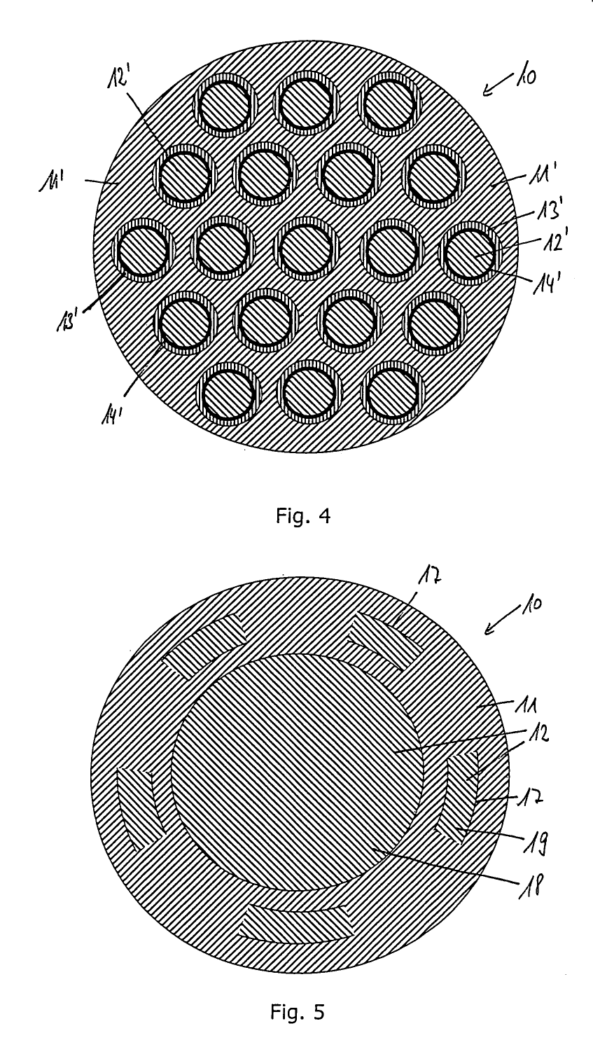

FIG. 4 shows a plan view of an electrode tip of an electrode according to the invention, in another preferred illustrative embodiment;

FIG. 5 shows a cross section through an electrode tip of an electrode according to the invention, in another preferred illustrative embodiment;

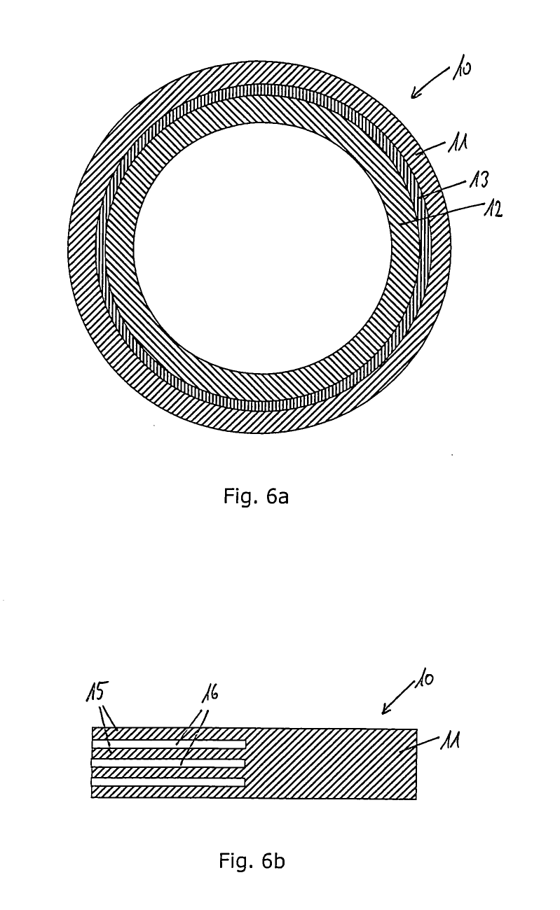

FIG. 6a shows a cross section through an electrode tip of an electrode according to the invention, in another illustrative embodiment;

FIG. 6b shows a side view of the electrode tip according to FIG. 6a;

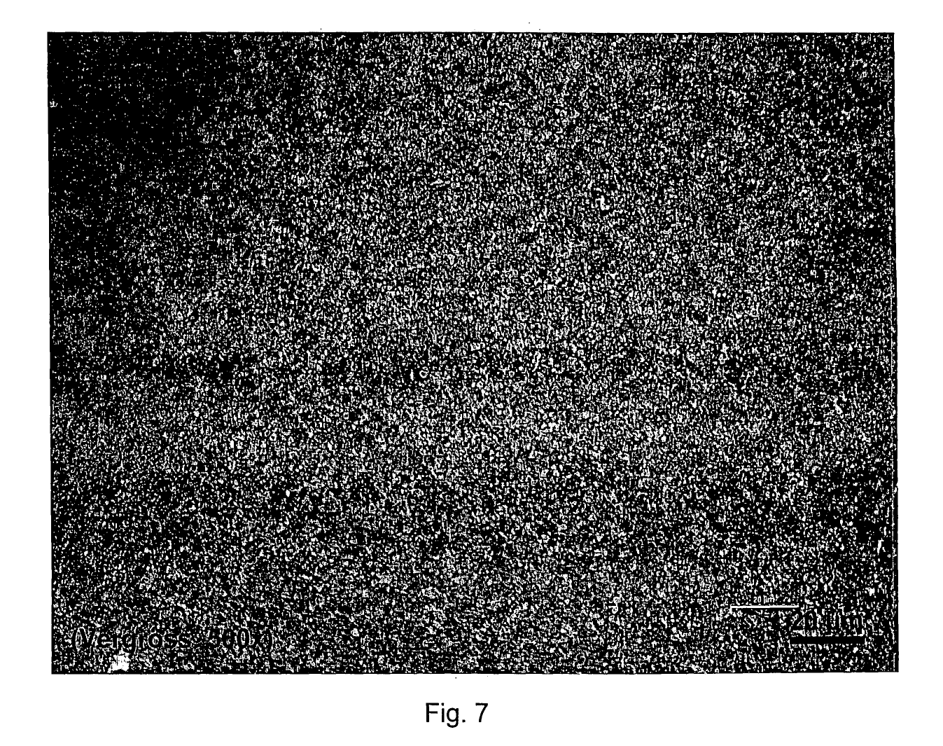

FIG. 7 shows a micrograph (500× magnification) of an electrode tip produced according to the invention, and

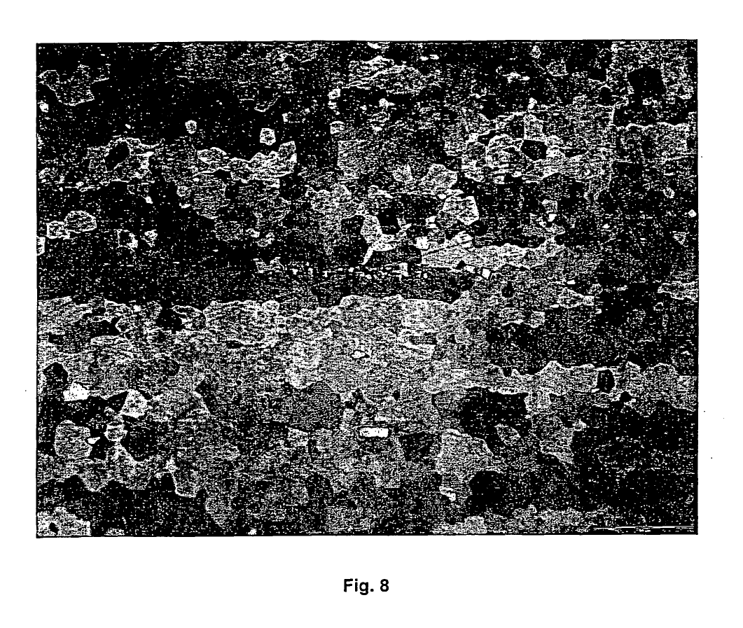

FIG. 8 shows a micrograph (500× magnification) of a conventionally produced electrode tip.

The illustrative embodiments described below are based principally on the same production method, wherein the electrode tip, in particular the support structure of the electrode tip, is produced by a vapor deposition method, in particular a sputtering method, from a shape-memory material. The support structure can comprise a hollow core for formation of a hollow electrode. Alternatively, the support structure can be made from solid material, i.e. without a hollow core, for formation of a solid electrode tip.

The entire support structure can be produced by the vapor deposition method. It is also possible to form a support structure composite of different materials and/or of differently produced materials. Thus, part of the support structure, for example an outer area of the support structure, can be produced by sputtering, and another part of the support structure, for example a core, can be produced by a conventional method, for example by reshaping. It is also possible to produce the inner area by sputtering and the outer area by a conventional method, for example by reshaping, and then to join the areas. By suitable choice of the materials, for example shape-memory materials for both areas, the flexibility of the support structure composite can be obtained. It is also possible to differently produce more than two different areas of the support structure and/or to use different materials.

It is possible to produce 30%-90% of the material of the support structure by the vapor deposition method. The lower limit of the range can be 35%, in particular 40%, in particular 45%, in particular 50%, in particular 55%, in particular 60%. The upper limit of the range can be 85%, in particular 80%, in particular 75%, in particular 70%, in particular 65%. The aforementioned range limits can be combined with one another.

The support structure or the sputtered area of the support structure has a thickness, in particular in the radial direction, or a wall thickness of at least 5 μm, in particular at least 10 μm, in particular at least 15 μm, in particular at least 20 μm, in particular at least 25 μm, in particular at least 30 μm, in particular at least 35 μm, in particular at least 40 μm, in particular at least 45 μm, in particular at least 50 μm, in particular at least 100 μm, in particular at least 200 μm, in particular at least 300 μm, in particular at least 400 μm, in particular at least 500 μm. This applies to each individual area that is sputtered, or also to the sum of the thickness of all the sputtered areas (for example if several materials are used). The upper limit of the wall thickness can be, for example, at most 5 mm, in particular at most 4.5 mm, in particular at most 4 mm, in particular at most 3.5 mm, in particular at most 3 mm, in particular at most 2.5 mm, in particular at most 2 mm, in particular at most 1.5 mm, in particular at most 1 mm, in particular at most 0.5 mm. The aforementioned upper limits are disclosed in connection with the aforementioned lower limits.

With small wall thicknesses, for example in the case of films having a thickness of at least 5 μm, in particular at least 10 μm, in particular at least 15 μm, in particular at least 20 μm, the stability of the support structure is achieved by the geometry of the electrode tip. For example, a film that is shaped as a hollow cylinder is sufficiently stable to be used as a support structure. A person skilled in the art determines the length and the diameter of the hollow cylindrical support structure in such a way as to obtain a sufficiently stable electrode tip.

The sputtering method can also be used for other materials that can form part of the electrode tip. In sputtering, or generally in ion-beam-assisted deposition, a combined thermal electron beam evaporation with simultaneous ion bombardment of a substrate is advantageous, wherein an inert gas, for example argon, xenon, nitrogen or neon, is used. By the bombardment with inert gas, for example argon ions, the lattice defect density in the material of the electrode tip can be reduced. Alternative deposition methods that can be used to produce the electrode tip 10 or, generally, the electrode include laser ablation and direct ion-beam deposition, and also deposition methods based on cathodic arc discharges. Vacuum deposition methods are particularly suitable for adjusting the mechanical properties of the electrode tip 10 through the properties of the material used, in particular the adjustment of the lattice defect density. By suitable treatment following the deposition method, for example by annealing or age-hardening, high-pressure treatment or quenching, in particular by means of gas, the mechanical properties can be improved and/or the lattice defect density can be further reduced directly.

A suitable control device is advantageously provided that controls the process parameters and accordingly influences the production method. The control device is designed in particular to monitor and to control or regulate the following process steps.

To influence the lattice defect density of the material of the electrode tip 10, various process parameters can be varied. In particular, the lattice defect density is dependent on the chamber pressure, the deposition pressure and the partial pressure of the process gas during the production and/or deposition of the material. Therefore, during the production method, parameters of this kind are continuously detected and compared with previously determined setpoint values, in order to continuously ensure the quality of the electrode tip 10 that is produced. In particular, the reactive and non-reactive gases that are used in the deposition methods are metered in a controlled and/or suitable manner. The inert or non-reactive gases used preferably comprise argon or nitrogen. The substrate on which the one or more layers of materials for the electrode tip 10 are deposited can be stationary or movable. For example, the substrate can be rotated about a longitudinal axis in order to produce a three-dimensional, sputtered electrode tip 10. A movable substrate is particularly suitable for producing cylindrical electrode tips 10 or, generally, for electrode tips 10 that have a curved and in particular rotationally symmetrical surface. Instead of or in addition to a rotation of the substrate, other movements of the substrate within a system of coordinates can be provided in order to present particular geometries for the electrode tip 10.

Generally, the material of the electrode tip 10 can be deposited uniformly on the substrate in order to form the support structure. Alternatively, the material can be deposited on the substrate in such a way that a structuring is obtained. The structuring can be defined by a lithography method, in particular a photo-lithography method.

The material deposited on the substrate can be detached from the substrate in various ways. For example, the deposited material can be removed from the substrate by chemical etching or chemical stripping or mechanically. It is also possible to provide a sacrificial layer between the substrate and the material for the electrode tip 10, which sacrificial layer is removed by chemical etching or melting or by other suitable methods in order to detach the produced electrode tip 10 from the substrate.

A hollow electrode is thus formed whose supporting structure or support structure is produced by the PVD method, in particular by the sputtering. The supporting structure determines the mechanical and electrical properties of the hollow electrode or the hollow electrode tip. The wall thickness of the hollow electrode tip can be small, for example at least 5 μm, in particular at least 10 μm, in particular at least 15 μm, in particular at least 20 μm, in particular at least 25 μm, in particular at least 30 μm, in particular at least 35 μm, in particular at least 40 μm, in particular at least 45 μm, in particular at least 50 μm, in particular at least 100 μm, in particular at least 200 μm, in particular at least 300 μm, in particular at least 400 μm, in particular at least 500 μm. The hollow electrode tip can be guided on a guide wire. It is also possible to provide a core in the electrode tip, which core is surrounded by a sputtered layer of a shape-memory material, the layer being so thick that it assumes a supporting function together with the core.

The size of the precipitations formed upon deposition of the target material is limited. Specifically, the upper limit is 500 nm. Lower upper limits are possible. Thus, the upper limit of the size of the precipitations can be 450 nm, in particular 400 nm, in particular 350 nm, in particular 300 nm, in particular 250 nm, in particular 200 nm, in particular 150 nm, in particular 100 nm, in particular 75 nm, in particular 50 nm, in particular 25 nm, in particular 20 nm. The lower limit can be 10 nm, in particular 15 nm, in particular 20 nm, in particular 25 nm, in particular 30 nm, in particular 35 nm, in particular 40 nm, in particular 45 nm, in particular 50 nm and can be combined with the aforementioned respectively higher upper limits. With decreasing size of the precipitations, the conductivity of the material is further improved, since the charge carriers are less scattered on the precipitations.

The specific adjustment of the size and/or density of lattice defects by the vapor deposition, in particular the sputtering, is another measure for improving the conductivity. This concerns zero-dimensional and/or unidimensional and/or two-dimensional and/or three-dimensional lattice defects. Zero-dimensional lattice defects are, for example, voids, interstitial atoms and foreign atoms. Unidimensional lattice defects include dislocations, in particular edge dislocations and screw dislocations. Two-dimensional lattice defects can be grain boundaries, stacking faults and twin boundaries. Three-dimensional lattice defects are pores, inclusions and precipitations. Since, in the case of shape-memory materials, precipitations are crucial for the properties of the shape-memory materials, limiting the size of the precipitations is a particularly effective measure. In the case of the other lattice defects, their density and/or if applicable their size is specifically adjusted. In addition to limiting the size of the precipitations, the density of the latter can also be specifically adjusted.

The electrode tip can be formed from a single layer (not shown) or from at least two layers 11, 12. This applies in particular if the electrode tip has a substantially cylindrical shape, in particular a hollow cylindrical or rotationally symmetrical shape. Alternatively, the electrode tip 10 can also form a planar structure, for example a plate or leaf. In the case of a planar structure, the electrode tip 10 can comprise at least two areas 11, 12 with different materials.

A possible basic structure of a substantially cylindrical electrode tip 10 with two different material layers 11, 12 is shown by way of example in FIG. 1. The support structure is in this case formed from a solid material. The basic structure can be extended to more than two layers.

FIG. 1 shows, as an example, the design of an electrode tip 10 that has a rotationally symmetrical shape and is formed from two layers 11, 12. The inner layer 12, or core layer, preferably comprises a material that has a higher conductivity than the outer layer 11, or jacket layer. For example, the inner layer 12 can comprise copper, silver, gold, platinum, tantalum, niobium, palladium or carbon. The inner layer 12 can be made available as a wire, for example, in the production of the electrode tip. During the production process, the wire can serve as a substrate for the outer layer 11 or jacket layer. The jacket layer or outer layer 11 can be deposited on the inner layer 12 by a sputtering process. The outer layer 11 and the inner layer 12 can merge smoothly or continuously into each other. This means that there are no clearly defined interfaces between the outer and inner layers 11, 12. Instead, a radial material gradient can be established in the electrode tip 10. The material gradient can be adjusted, for example, by controlling a number of targets with different compositions, such that the material composition of the electrode tip 10 changes continuously.

The inner layer 12 can further comprise a material that ensures increased X-ray visibility. In this way, the position of the electrode tip 10 within the living body can be monitored by an imaging method, in particular based on X-radiation.

The outer layer 11 preferably comprises a shape-memory material. The shape-memory material has a crystalline structure, wherein the inhomogeneities in the crystal structure are specifically adjusted, in particular by varying the parameters of the sputtering process during production. The measures necessary for this are determined, for example experimentally, by a person skilled in the art. The outer layer 11 preferably comprises a nickel-titanium alloy, in particular nitinol. Generally, the outer layer 12 has mechanical properties in combination with electrically conductive properties. By contrast, the inner layer 12 mainly has conductive properties in combination with X-ray visibility properties. The aforementioned features apply to all the illustrative embodiments.

A further illustrative embodiment of the support structure of an electrode tip is shown in FIG. 2, in which an intermediate layer 13 is arranged between the outer layer 11 and the inner layer 12. The intermediate layer 13 can have insulating properties, for example. In particular, the intermediate layer 13 can comprise an insulating plastic material. In this way, various signals or electric currents can be transferred through the electrode tip 10. For example, a bipolar electrode tip 10 can be made available with the aid of the insulating intermediate layer 13. It is also possible that one of the layers 11, 12, for example the outer layer, is used to pick up electrical signals from the body tissue, for example for measuring the electrical activity of the body tissue, whereas the inner layer is used to deliver an electric current to the body tissue, for example for the purpose of stimulating the body tissue.

Specifically, the outer layer 11 can be used to detect for example electrical signals of the heart muscle, in particular of the pacemaker cells. The measured electrical signals can be evaluated using a suitable measuring device. The inner layer 12 is used to introduce a current impulse into the heart muscle cells, wherein the current impulse is generated according to the measurement result of the signals transferred via the outer layer 11. Such a design of the electrode tip 10 is advantageous, for example, in electrodes for heart pacemakers or for heart pacemaker/defibrillator devices. The possibility of the materials and/or functions of the layers 11, 12 being reversed is not excluded. For example, the outer layer 11 can have a material with increased electrical conductivity, for example for stimulation of body cells, and the inner layer 12 can comprise a shape-memory material that combines predominantly mechanical properties with electrically conductive properties, the inner layer 12 being used to detect electrical signals of the body cells.

Advantageously, at least the outer layer 11 and/or the intermediate layer 13 and/or the inner layer 12 are produced by a sputtering method. A relatively small wall thickness is thus achieved, wherein the mechanical properties, in particular the flexibility, of the electrode tip 10 are increased on account of the pseudo-elastic properties of the shape-memory material. This applies in particular to the insulating intermediate layer 13, which can have a comparatively thin wall, without the insulating properties being impaired. In particular, the relatively homogeneous structure of the sputtered intermediate layer 13 has the effect that the insulating properties are maintained even if the flexible electrode tip 10 is subjected to a high bending stress.

It is important to emphasize generally the dual function of the shape-memory material, on the one hand as an electrical conductor and, on the other hand, as a supporting material that determines the mechanical properties of the electrode tip 10. This means that the strength of the material area formed from the shape-memory material is dimensioned in such a way that it assumes a supporting function. This is the case, for example, in a hollow electrode whose wall is formed from the shape-memory material.

Alternatively to a cylindrical design of the flexible electrode tip 10, the electrode tip 10 can have a planar or flat shape, as is shown in FIGS. 3a and 3b. It is possible to produce the planar or flat shape as a single layer (not shown). The planar structure is composed of a sputtered solid material. In the case of two layers, outer and inner areas 11′, 12′ are provided instead of the outer and inner layers 11, 12, which areas have substantially the same properties as the outer and inner layers 11, 12 according to the above-described illustrative embodiments. The basic structure with two areas can be transposed to other structures with more than two areas. In the cross-sectional view according to FIG. 3a, it will be seen that the outer and inner areas 11′, 12′ can alternate. Here, the inner areas 12′ can be embedded in a basic structure of the outer area 11′, as can be seen from FIG. 3b. The basic structure of the outer area 11′ forms the support structure. Preferably, the inner areas 12′ are characterized by an electrically particularly conductive material, whereas the basic material of the outer area 11′ comprises a shape-memory material that importantly influences the increased flexibility of the electrode tip 10. Overall, the flexible electrode tip 10 according to FIG. 3b can have a circular, plate-shaped structure, in which the main body of the flexible electrode tip 10 is formed by the outer areas 11′ comprising the shape-memory material. In the outer area 11′, a plurality of inner areas 12 are embedded that have a different material, in particular a material with an electrical conductivity that exceeds the conductivity of the material of the outer area 11′.

The plate-shaped electrode tip 10 can be structured in different ways. In the illustrative embodiment according to FIGS. 3a and 3b, the plate-shaped flexible electrode tip 10 has a substantially spotted structure. It is also possible that the electrode tip 10 has a structure substantially similar to a printed circuit board, for example in the manner of an electronic circuit board. Other types of structuring are possible. In particular, the electrode tip 10 can have a three-dimensional structuring, in which case, for example, the inner areas 12′ can protrude above the outer area 11′.

As is shown in FIG. 4, the plate-shaped electrode tip 10 can have further areas, in particular an intermediate area 13′ and a separating area 14′. The separating area 14′ and the intermediate area 13′ can be arranged concentrically surrounding the inner area 12′. Like the intermediate layer 13 in the cylindrical electrode tip 10 according to FIG. 2, the intermediate area 13′ has an electrically insulating material. The intermediate area 13′ surrounds the inner area 12′ and the separating area 14′ in a ring shape. The intermediate area 13′ thus insulates the separating area 14′ from the outer area 11′. The separating area 14′ can likewise comprise an insulating or an electrically conductive material. Preferably, the separating area 14′ has an electrically conductive material.

It is advantageous to produce the planar or flat electrode tip 10 by a sputtering process. The planar structure of the outer area 11′ can be structured after the sputtering, in order to separate the individual areas 11′, 12′, 13′, 14′ from one another. The structuring can, for example, involve a lithographic process. The planar structure of the electrode tip 10, in particular of the outer area 11′, can be etched by wet chemistry. Between the individual areas 11′, 12′, 13′, 14′, area boundaries can be formed that are arranged substantially perpendicularly with respect to the surface of the electrode tip 10. When the electrode tip 10 is structured by a wet chemical etching method, it is also possible to form area boundaries that are arranged at an inclination. The planar or flat electrode tip 10 can be completely covered or sputtered with an oxide. Alternatively or in addition, the electrode tip 10 can have a plastic layer onto which the individual areas 11′, 12′, 13′, 14′ are deposited. The plastic layer or the outer area 11′ can form a sputter base, which serves as substrate during the sputtering process. By means of a galvanic process, the insulating material of the base can grow into the interstices between the individual electrically conductive areas 12′, 13′, 14′. Generally, several different materials can be provided that are either insulated from one another by additional areas arranged between them or form an electrical contact with one another.

It is also possible for the plate to be produced from a single material, in particular from a single shape-memory material, by a PVD method.

The cylindrical shape or, generally, the three-dimensional shape of the electrode tip can be formed, for example, by heat treatment, such that the shape of the electrode is obtained from the shape-memory effect. Alternatively, the three-dimensional shape of the electrode can be produced by reshaping the planar electrode tip, in particular by rolling the electrode tip. In this case, the flat electrode tip 10 forms the preliminary product or an intermediate product prior to the final production of the cylindrical or three-dimensional electrode tip.

In the illustrative embodiment according to FIG. 4, provision is advantageously made that the outer area 11′ and the separating area 14′ have substantially insulating properties. The inner area 12′ and the intermediate area 13′ each comprise electrically conductive materials. The electrically conductive areas 12′, 13′ are arranged concentrically with respect to one another on or in the structure of the outer area 11′, which has a substantially insulating action. Between the inner area 12′ and the intermediate area 13′, there is a concentrically arranged separating area 14′, which comprises an insulating material and electrically separates the electrically conductive inner area 12′ from the electrically conductive intermediate area 13′. With this arrangement, it is possible, for example, that the inner area 12′ transfers a current into the body tissue in order to stimulate body cells and that the intermediate area 13′ can be used to pick up electrical signals from body cells. Alternatively, both electrically conductive areas 12′, 13′ can each form a respective pole of a bipolar electrode tip 10.

Alternatively, the outer area 11′ can be electrically conductive and separated from the intermediate area 13′ by another area.

FIG. 5 shows an electrode tip 10 that has a substantially cylindrical shape. The electrode tip 10 comprises several material layers 11, 12, wherein the inner layer 12 on the one hand forms the core of the electrode tip 10 and on the other hand is partially embedded in the outer layer 11. The inner layer 12 comprises an electrically conductive material. An insulating material which, in the illustrative embodiment according to FIG. 5, forms the outer layer 11 can be sputtered or deposited onto the core formed by the inner layer 12. In a further step, a further inner layer 12 is sputtered onto the insulating material of the outer layer 11, wherein the inner layer 12 is structured. On the periphery of the insulating outer layer 11, this results in areas of the inner layer 12 that are spaced apart from one another and are oriented substantially in the longitudinal direction of the electrode tip 10. In a further step, the outer layer is deposited again, such that the gaps between the areas of the inner layer 12 distributed on the periphery are closed and the inner layer 12 is completely enclosed by the outer layer 11. Overall, the outer layer 11 therefore completely surrounds, on the one hand, the core of the electrode tip 10 formed by the inner layer 12 and, on the other hand, the separate areas spaced radially apart from the core, which areas are likewise formed by the inner layer 12. The areas of the inner layer 12 that are spaced radially apart from the core form substantially a structured ring 19. This means that the areas of the inner layer 12 that are spaced apart from the core have a hollow cylindrical shape, wherein the hollow cylindrical shape is interrupted by openings, pores, holes or slits. The structured ring 19 therefore has gaps.

It is possible that the radial outer faces 17 of the areas formed by the inner layer 12 and spaced apart from one another in the circumferential direction are exposed. This means that the radial outer faces 17 are not covered by the outer layer 11. Instead, the radial outer faces 17 can be arranged on the outer circumference of the electrode tip 10, such that electrical signals can be detected or output across the outer surface of the electrode tip 10. In this case, the insulating outer layer 11 fills the space between the core 18 and the structured ring 19 and also the gaps in the structured ring 19. The structured ring 19 forms, together with the filled gaps, the outer surface of the electrode tip 10.

The electrode tip 10 can also have a hollow cylindrical shape or design, as is shown in FIG. 6a. The hollow cylinder, which forms the support structure of the electrode tip 10, has an outer layer 11, an inner layer 12 and an intermediate layer 13, which extends between the outer layer 11 and the inner layer 12. The outer layer 11, the inner layer 12 and the intermediate layer 13 are arranged concentrically with respect to one another. The inner layer 12 defines an internal diameter of the electrode tip 10. The outer layer defines an external diameter of the electrode tip 10. The outer layer 11 preferably has a shape-memory material. The inner layer 12 preferably has a material with a higher conductivity than the outer layer 11. Conversely, the inner layer 12 can be formed by a shape-memory material. The outer layer 11 can have an increased conductivity. The intermediate layer 13 is preferably made of an insulating material.

It is also possible that all the layers are electrically conductive and are separated by separating materials.

The electrode tip 10 produced in several layers by a sputtering method or generally by a deposition method can then be structured. For example, by means of a laser-cutting method, the hollow cylindrical electrode tip 10 according to FIG. 6a can be provided with recesses 16, as is shown in FIG. 6b. In particular, several recesses 16 can be introduced in the area of an axial end of the electrode tip 10, such that arms 15 form. The recesses 16 are preferably spaced apart uniformly over the circumference of the electrode tip 10 and have a uniform width. This results in arms 15 that are uniformly wide and spaced apart from one another, and these arms 15 can be used, for example, to anchor the electrode tip 10 in the body tissue. The arms 15 can extend rectilinearly in the longitudinal direction of the electrode tip 10. It is also possible that the arms 15 are curved, in particular in the radial direction. The arms 15 can also be bent in the circumferential direction of the electrode tip 10. In particular, the arms 15 can be wound in a spiral shape about the longitudinal axis of the electrode tip 10, in particular wound in a spiral shape.

FIG. 7 illustrates the small grain sizes that are obtainable by deposition compared with conventionally produced electrode tips (FIG. 8). The uniform distribution of the grains (FIG. 7) in contrast to the tiered arrangement of the grains (FIG. 8) will also be noted.

All the features of the aforementioned illustrative embodiments, which are disclosed in connection with the electrode or the electrode tip, are also disclosed in connection with the method for producing the electrode or the electrode tip, and vice versa. Moreover, all the individual features of the illustrative embodiments can be combined with one another.

### LIST OF REFERENCE SIGNS

10 electrode tip
11 outer layer
11′ outer area
12 inner layer
12′ inner area
13 intermediate layer
13′ intermediate area
14 separating layer
14′ separating area
15 arm
16 recess
17 radial outer face
18 core
19 structured ring

1. An electrode for measuring electrical activities of a living body and/or for outputting electrical signals into the living body and/or for ablating tissue or nerve paths, with an electrically insulated and flexible line which is connected fixedly or releasably to a distally arranged and electrically conductive electrode tip, the electrode tip being designed for insertion into the body, wherein at least one support structure of the electrode tip comprises at least one crystalline shape-memory material, and the electrode tip is flexible on account of the shape-memory material, wherein the shape-memory material of the support structure is formed at least partially by physical vapor deposition, in such a way that precipitations in the shape-memory material have a maximum size of 500 nm.

2. The electrode as claimed in claim 1, wherein the shape-memory material of the support structure has a maximum density of material inhomogeneities, such as lattice defects, that is specifically adjusted by physical vapor deposition.

3. The electrode as claimed in claim 1, wherein the grain size of the shape-memory material of the support structure is less than 4 μm, in particular less than 3 μm, in particular less than 2 μm, in particular less than 1.5 μm, in particular less than 1 μm, in particular less than 0.5 μm, in particular less than 250 nm.

4. The electrode as claimed in claim 1, wherein the shape-memory material of the support structure is produced at least partially by sputtering or by laser-assisted deposition.

5. The electrode as claimed in claim 1, wherein the shape-memory material comprises a nickel-titanium alloy, in particular with a composition of 50.8 at % nickel and 49.2 at % titanium.

6. The electrode as claimed in claim 1, wherein the shape-memory material comprises a ternary or quaternary alloy.

7. The electrode as claimed in claim 1, wherein the electrode tip has a multi-layer configuration.

8. The electrode as claimed in claim 7, wherein a radially outer layer or several radially outer layers are composed of the shape-memory material, in particular of a nickel-titanium alloy, and a radially inner layer or several radially inner layers are composed of a material with higher electrical conductivity than the material of the radially outer layer or radially outer layers, in particular of copper, silver, gold, platinum, tantalum, niobium, palladium or carbon.

9. The electrode as claimed in claim 7, wherein a radially continuous transition is present between the layers.

10. The electrode as claimed in claim 1, wherein the radially inner layer or the radially inner layers are composed of a nickel-titanium alloy and the radially outer layer or the radially outer layers are composed of a material with higher electrical conductivity than the material of the radially inner layer or radially inner layers.

11. A method for producing an electrode for measuring electrical activities of a living body and/or for outputting electrical signals into the living body, with an electrically insulated and flexible line which is connected fixedly or releasably to a distally arranged and electrically conductive electrode tip, the electrode tip being designed for insertion into the body, in which method at least one support structure of the electrode tip is formed at least partially from at least one crystalline shape-memory material by physical vapor deposition, in such a way that the electrode tip is flexible on account of the shape-memory material.

12. The method as claimed in claim 11, wherein several layers of different materials are applied for forming the electrode tip, wherein the radially inner layers and the radially outer layers are produced by a combination of a lithography method and a sputtering method.
